(19) United States
(12) Patent Application Publication
(10) Pub. No.: US 2017/0143868 A1
(43) Pub. Date: May 25, 2017

(54) ATTACHABLE AND MOVABLE DEODORIZER FOR ENCLOSED SPACE

(71) Applicant: CONARY ENTERPRISE CO., LTD., TAIPEI CITY (TW)

(72) Inventors: SHIH JUNG DANICA HUANG, TAIPEI CITY (TW); CHIH LIANG LIN, TAIPEI CITY (TW)

(21) Appl. No.: 14/947,097

(22) Filed: Nov. 20, 2015

(51) Int. Cl. A61L 9/20; A61L 9/16

(52) U.S. Cl. CPC A61L 9/205; A61L 9/16

(57) ABSTRACT

An attachable and movable deodorizer for enclosed space has a body with a plurality of inlet holes and at least one outlet hole. The body has a fan, a photocatalyst filter, and an ultraviolet (UV) control circuit board connected to an UV-A LED lamp. Whereby the body is able to be disposed in an enclosed space such as trash bin and shoes cabinet, and to suck in the dirty air in the enclosed space by the fan for the dirty air to pass through the photocatalyst filter and then to be discharged from the outlet hole. The stench chemical substance in the dirty air is therefore purified in the photocatalysis process.

### BACKGROUND OF THE INVENTION

1. Field of the Invention

The present invention relates to a deodorizer, particularly to one that can be attached within and removed from an enclosed space and one that has an ultraviolet-A (UV-A) LED lamp to illuminate a photocatalyst filter for photocatalysis and therefore purify the dirty and stench air in the enclosed space.

2. Description of the Related Art

In spaces with poor air circulation, there are often bacteria, dusts, and suspended substance in the air; in some enclosed spaces such as shoes cabinets and trash bins, it is even worse—thriving bacteria and stench smell spreading all over. An ultraviolet-C (UV-C) has a wavelength between 200-280 nm. It can directly destroy the DNA structure of bacteria, virus, and microorganism to stop them from thriving and inhabitation. FIG. 1 illustrates an UV-C lamp 300 disposed inside a shoes cabinet 200. When a pair of shoes is put inside the shoes cabinet 200 as in an enclosed space 90, the bacteria would thrive in the enclosed space 90 due to poor air circulation and therefore the enclosed space 90 will be filled with stench smell. The UV-C lamp 300 is able to destroy the bacteria but cannot deal with the stench smell, not to mention that it can cause damages to human body upon exposure.

Photocatalyst is developed from UV lighting; it is a series of chemical reaction as the energy of lights absorbed for the catalysis process with titanium dioxide (TiO₂) nanoparticles being the catalyst. The photocatalytic activity depends on the ability of the catalyst to create electrons and electron-holes, which generate hydroxyl radicals OH⁻ to decompose the organic molecules, so as to easily turn the stench chemical substance in the air into clean and harmless substance. As a result, photocatalyst is able to deodorize, prevent bacteria and mildew from thriving, and decompose dirt. FIG. 2 shows a LED lamp 310 and a photocatalyst 400 disposed inside a trash bin 500. The photocatalyst 400 is spread on the inner surface of a cover 510 of the trash bin 500. With the trash inside the trash bin 500 as in an enclosed space 90, the bacteria would rapidly thrive due to poor air circulation and therefore produce a stench smell. The air near the photocatalyst 400 can be decomposed and deodorized, but deep down the trash bin 500 the air far from the photocatalyst 400 cannot be processed since the illumination area of the photocatalyst 400 is limited. In other words, spreading photocatalyst 400 on the inner surface of the cover 510 can only deal with partial dirty air within the trash bin 500.

To sum up, the UV-C lamp 300 cannot fully deodorize the air within the enclosed space 90 of the shoes cabinet 200, and the photocatalyst 400 spread on the cover 510 of the trash bin 500 with a LED lamp 310 cannot entirely purify the air filled within the enclosed space 90 of the trash bin 500 either. Hence, it is desirable to provide a device that is conveniently installed and that purifies the dirty and stench air within an enclosed space without potential damages to human body.

### SUMMARY OF THE INVENTION

A primary object of the present invention is to provide an attachable deodorizer for enclosed space to purify the air in the enclosed space by a photocatalyst filter.

Another object of the present invention is to provide an attachable and easily removable deodorizer for enclosed space such as trash bin and shoes cabinet.

Yet another object of the present invention is to provide a deodorizer disposed in an enclosed space that is able to instantly cease the operation when a door thereof is opened, preventing potential damages to human body.

In order to achieve the object above, the present invention comprises a body having a placing space therein, a plurality of inlet holes arranged at the front thereof, and at least one outlet hole arranged aside; a fan disposed inside the placing space near the inlet holes; a photocatalyst filter disposed at the rear of the fan for the air sucked in by the fan to pass through the photocatalyst filter; an ultraviolet (UV) control circuit board including an UV-A LED lamp, a microcontroller, and a controlling chip; said UV control circuit board being disposed in the placing space and electrically connected to the fan for the UV-A LED lamp to illuminate and catalyse the photocatalyst filter for deodorization; a wire connected to the UV control circuit board with an end to supply electricity for the UV control circuit board and the fan; whereby when the body is disposed in an enclosed space of a trash bin or a shoes cabinet with a movable member for opening, the dusts and suspended substance filled within would be sucked into the body via the inlet holes by the fan, pass through the photocatalyst filter, and then be discharged from the body via the outlet hole; and the photocatalyst filter is able to purify the dirty air with stench chemical substance within the enclosed space.

Furthermore, at least one attaching piece being a magnet, a touch fastener, or a double-sided adhesive is arranged at the corresponding side of the inlet holes for the body to be attached and removed in an enclosed space such as a trash bin and a shoes cabinet. The controlling chip has time settings of repeated activating and standing-by for the fan and the UV-A LED lamp to operate periodically. The present invention further includes a sensor disposed on the body and electrically connected to the microcontroller for detecting status of the movable member so that when the movable member is opened, the controlling chip is able to stop the operation of the fan and the UV-A LED lamp, and when the movable member is closed, the controlling chip is able to activate the operation, and includes a manual switch disposed on the body and electrically connected to the microcontroller so that when the air in the enclosed space is too stench, pressing the manual switch can reset the operation period of the fan and the UV-A LED lamp and reactivate the operation.

The wire has the other end thereof connected to a battery device that has normal batteries or rechargeable batteries desposed therein. The present invention further includes a filter to be disposed between the inlet holes and the fan to filter the dusts, hairs, etc. The UV-A LED lamp has a wavelength between 360-400 nm, preferably 380 nm.

With features disclosed above, the present invention has the fan to suck the dirty air within the enclosed space into the body and purify the stench chemical substance in the dirty air by having an UV-A LED lamp illuminating and catalysing the photocatalyst filter. Also, the present invention has at least one attaching piece which is a magnet, a touch fastener, or a double-sided adhesive for the device to be easily attached and removed within the enclosed space. In addition, the sensor thereof is electrically connected to the microcontroller for opening detection so that the controlling chip is able to stop the operation of the fan and the UV-A LED lamp when the space is opened, preventing potential damages to human body.

### BRIEF DESCRIPTION OF THE DRAWINGS

FIG. 1 is a schematic diagram of a conventional shoes cabinet with an ultraviolet-C (UV-C) lamp therein;

FIG. 2 is a schematic diagram of a conventional trash bin with photocatalyst therein;

FIG. 3 is an exploded view of the present invention;

FIG. 4 is a perspective view of the present invention;

FIG. 5 is a sectional view of the present invention;

FIG. 6 is a circuit diagram of the present invention;

FIG. 7A is a schematic diagram of the present invention applied to a shoes cabinet; and

FIG. 7B is another schematic diagram of the present invention applied to a shoes cabinet.

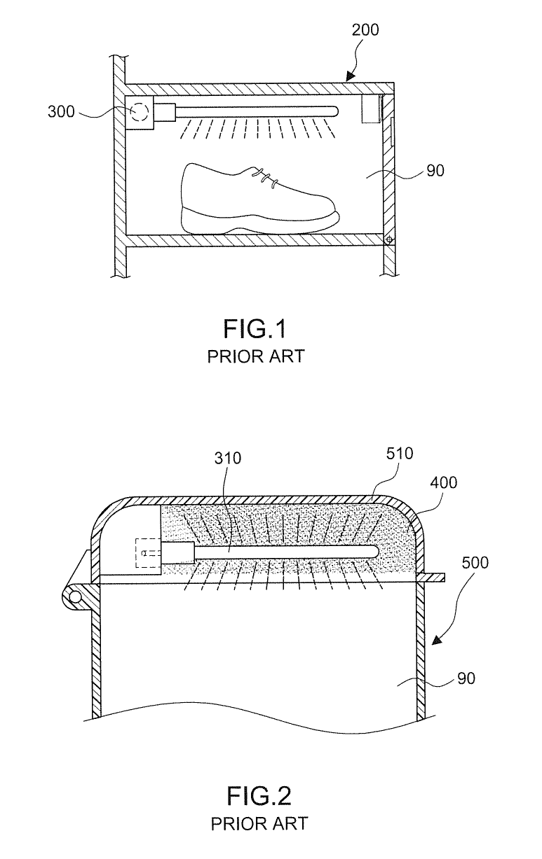

### DETAILED DESCRIPTION OF THE PREFERRED EMBODIMENT

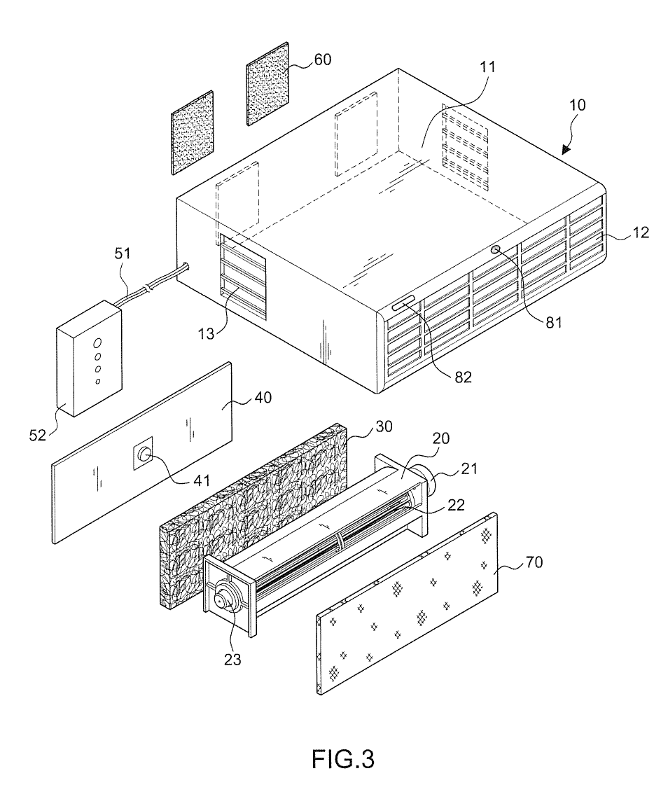

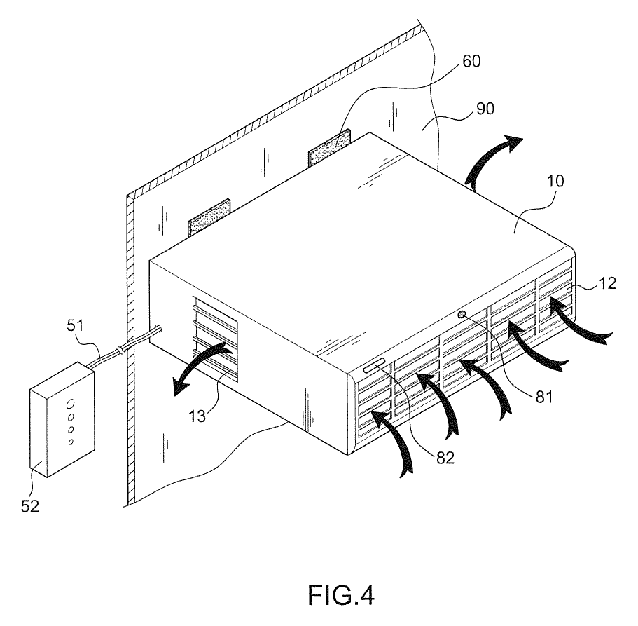

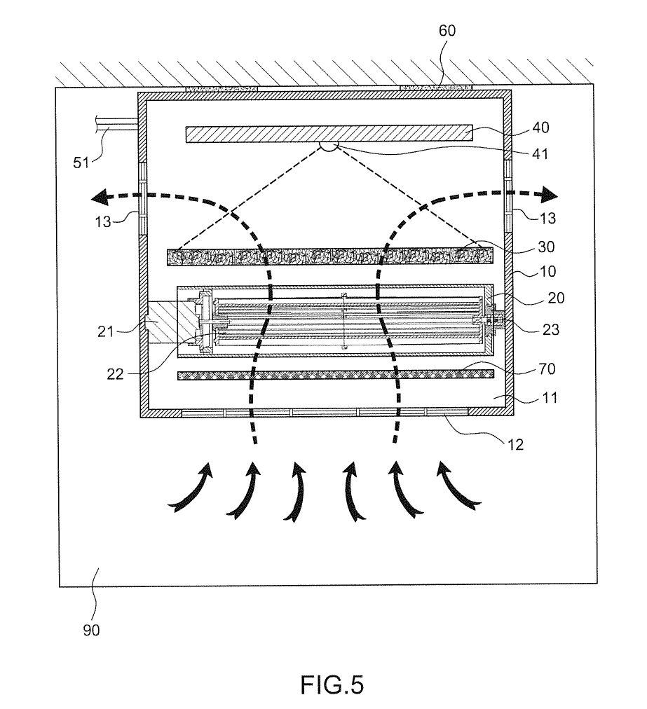

Referring to FIGS. 3-5, in a preferred embodiment, the present invention mainly includes a body 10, a fan 20, a photocatalyst filter 30, an ultraviolet (UV) control circuit board 40, and a wire 51.

The body 10 has a placing space 11 therein, a plurality of inlet holes 12 arranged at the front thereof, and at least one outlet hole 13 arranged aside. The fan 20 is disposed inside the placing space 11 near the inlet holes 12. The photocatalyst filter 30 is disposed at the rear of the fan 20 for the air sucked in by the fan 20 to pass through the photocatalyst filter 30. The UV control circuit board 40 includes an UV-A LED lamp 41, a microcontroller 42, and a controlling chip 43. The UV control circuit board 40 is disposed in the placing space 11 and is electrically connected to the fan 20 for the UV-A LED lamp 41 to illuminate and catalyse the photocatalyst filter 30 for deodorization. The wire 51 is connected to the UV control circuit board 40 with an end to supply electricity for the UV control circuit board 40 and the fan 20.

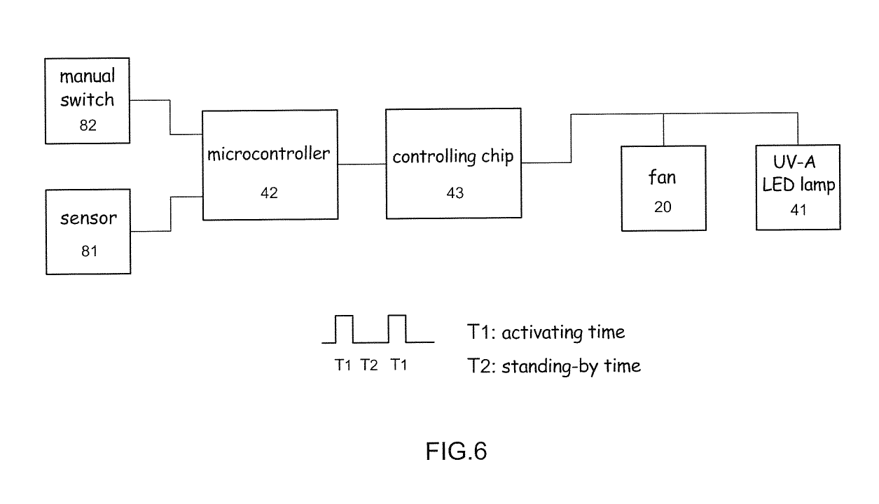

In this embodiment, at least one attaching piece 60 is arranged at the corresponding side of the inlet holes 12 for the body 10 to be attached and easily removed in an enclosed space 90 such as a trash bin and a shoes cabinet. The attaching piece 60 is a magnet, a touch fastener, or a double-sided adhesive, but it is not limited to such application. In addition, the present invention further includes a filter 70, a sensor 81 and a manual switch 82. The filter 70 is disposed between the inlet holes 12 and the fan 70 for filtering substance such as dusts and hairs in the air before the air enters the device. The sensor 81 is disposed on the body 10 and is electrically connected to the microcontroller 42 as shown in FIG. 6. The controlling chip 43 has been set an activating time T1 and a standing-by time T2 to control the periodical operation of the fan 20 and the UV-A LED lamp 41. The sensor 42 is designed to detect status of a movable member 210 for opening so that when the movable member 210 is opened, the microcontroller 42 would send an instruction for the controlling chip 43 to cease the operation of the fan 20 and UV-A LED lamp 41; and when the movable member 210 is closed, the microcontroller 42 would send an instruction for the controlling chip 43 activate the operation. The manual switch 82 is also disposed on the body 10 and electrically connected to the microcontroller 42. When the stench smell in the air within the enclosed space 90 is too strong, a user can simply press the manual switch 82 to reset the operation period of the fan 20 and the UV-A LED lamp 41 and reactivate the operation. In the embodiment, the wire 51 further has the other end thereof connected to a battery device 52 that has normal batteries or rechargeable batteries disposed therein.

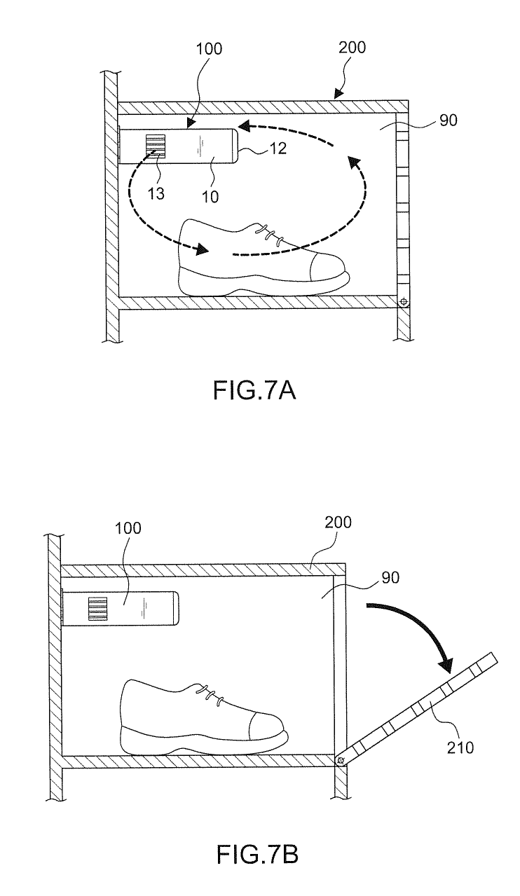

FIG. 7A illustrates the present invention, a deodorizer 100, being attached in a shoes cabinet 200. When a pair of shoes is put inside the enclosed space 90 of the shoes cabinet 200, the bacteria in the shoes would thrive within by the dusts and suspended substance, and even produce stench smell. The deodorizer 100 of the present invention is able to filter the dirty and stench air sucked into the body 10 thereof by the fan 20 and purify the chemical substance in the air by the photocatalyst filter 30 via a photocatalysis process with the illumination of the UV-A LED lamp 41, turning into clean and harmless substance and then discharging the clean air from the outlet hole 13. FIG. 7B further shows when the opening member 210, i.e. the door, of the shoes cabinet 200 is opened, the deodorizer 100 of the present invention has the sensor 81 to detect the opening status and to send instructional signals to the controlling chip 43 from the microcontroller 42 for instantly ceasing the operation of the UV-A LED lamp 41 and the fan 20, thus preventing from potential damages to human body.

With aforesaid structures and measures, the present invention is able to purify the air in the enclosed space 90 with the photocatalyst filter 30 and to instantly stop the operation for preventing damages from human body. Besides, it is attachable to other devices with similar enclosed space as the trash bin and shoes cabinet and is also easily removable, making it a convenient device for users.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. An attachable and movable deodorizer for enclosed space comprising:
a body having a placing space therein, a plurality of inlet holes arranged at the front thereof, and at least one outlet hole arranged aside;
a fan disposed inside the placing space near the inlet holes;
a photocatalyst filter disposed at the rear of the fan for the air sucked in by the fan to pass through the photocatalyst filter;
an ultraviolet control circuit board including an ultraviolet-A LED lamp, a microcontroller, and a controlling chip; said ultraviolet control circuit board being disposed in the placing space and electrically connected to the fan for the ultraviolet-A LED lamp to illuminate and catalyse the photocatalyst filter for deodorization;
a wire connected to the ultraviolet control circuit board with an end to supply electricity for the ultraviolet control circuit board and the fan;
whereby when the body is disposed in an enclosed space of a trash bin or a shoes cabinet with a movable member for opening, the dusts and suspended substance filled within would be sucked into the body via the inlet holes by the fan, pass through the photocatalyst filter, and then be discharged from the body via the outlet hole; and the photocatalyst filter is able to purify the dirty air with stench chemical substance within the enclosed space.

2. The attachable and movable deodorizer for enclosed space as claimed in claim 1, wherein at least one attaching piece being a magnet, a touch fastener, or a double-sided adhesive is arranged at the corresponding side of the inlet holes for the body to be attached and removed in an enclosed space such as a trash bin and a shoes cabinet.

3. The attachable and movable deodorizer for enclosed space as claimed in claim 1, wherein the controlling chip has time settings of repeated activating and standing-by for the fan and the ultraviolet-A LED lamp to operate periodically.

4. The attachable and movable deodorizer for enclosed space as claimed in claim 1, wherein the present invention further includes a sensor disposed on the body and electrically connected to the microcontroller for detecting status of the movable member so that when the movable member is opened, the controlling chip is able to stop the operation of the fan and the ultraviolet-A LED lamp, and when the movable member is closed, the controlling chip is able to activate the operation.

5. The attachable and movable deodorizer for enclosed space as claimed in claim 4, wherein the present invention further includes a manual switch disposed on the body and electrically connected to the microcontroller so that when the air in the enclosed space is too stench, pressing the manual switch can reset the operation period of the fan and the ultraviolet-A LED lamp and reactivate the operation.

6. The attachable and movable deodorizer for enclosed space as claimed in claim 1, wherein the wire has the other end thereof connected to a battery device that has normal batteries or rechargeable batteries disposed therein.

7. The attachable and movable deodorizer for enclosed space as claimed in claim 1, wherein the present invention further includes a filter to be disposed between the inlet holes and the fan.

8. The attachable and movable deodorizer for enclosed space as claimed in claim 1, wherein the ultraviolet-A LED lamp has a wavelength between 360-400 nm.

9. The attachable and movable deodorizer for enclosed space as claimed in claim 8, wherein the wavelength of the ultraviolet-A LED lamp is preferred to be 380 nm.
